(19) United States
(12) Patent Application Publication
(10) Pub. No.: US 2024/0335137 A1
(43) Pub. Date: Oct. 10, 2024

(54) BREATHALYZER SYSTEM FOR DETECTION OF RESPIRATORY PATHOGENS

(71) Applicant: Janus-l Science Inc., Vista, CA (US)

(72) Inventors: James C. Hannis, Vista, CA (US); Thomas A. Hall, Vista, CA (US); Lisa M. Risen, Vista, CA (US); Christine V. Marzan, Vista, CA (US); Michael R. Mosel, Vista, CA (US); David J. Ecker, Vista, CA (US)

(21) Appl. No.: 18/579,273

(22) PCT Filed: Jul. 15, 2022
(86) PCT No.: PCT/US2022/037332

§ 371 Date: Jan. 12, 2024

Related U.S. Application Data
(60) Provisional application No. 63/222,836, filed on Jul. 16, 2021.

(51) Int. Cl. A61B 5/08; A61B 10/00; G01N 21/64

(52) U.S. Cl. CPC A61B 5/082; A61B 10/0051; G01N 21/6408; G01N 21/6428

(57) ABSTRACT

Disclosed herein are sample and assay breathalyzer cartridges for sample collection and performance of assay composed of multiple layers and devices therefor.

The invention described herein was made with U.S. Government support under the Intelligence Advanced Research Projects Activity (IARPA) under Grant Number 2021-2012040000] awarded by the Office of the Director of National Intelligence Department of Veterans Affairs to James C. Hannis, Ph.D. The United States Government has certain rights in the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

The testing of respiratory patbogens has been associated with nasal wasbes, nasal aspirates, nasal swabs, nasopharyngeal swabs, pasopharyngeal washes, throat swabs or sputum that can be considered an invasive form of sampling. This type of sampling is used with both immunological and molecular type testing. Immunological point-of-care assays for respiratory diseases can be relatively quick (i.e., 15 to 20 minutes), however, they can be prone to false positives if inappropriately used, and lack the sensitivity of molecular assays that rely on amplification. Molecular assays for respiratory pathogens are typically not fieldable and are situated in laboratory settings or as bench top devices in doctors' offices-usually taking longer for amplification, detection and analysis than the immunological assays. Presently. there is a need for a portable system that can non-invasively capture a respiratory sample then automatically process, amplify and detect an agent without user intervention in a sensitive and timely manner.

A much less invasive method to capture respiratory pathogens would be to capture the pathogens as they are expelled with breath aerosols. Expelled breath aerosols have been shown to contain both viral and bacterial pathogens. However, no such commercial device has been shown to successfully perform respiratory pathogen detection from breath samples. Thus, a diagnostic platform that can capture and detect respiratory pathogens in a fieldable and point-of-care setting with molecular amplification sensitivity and a quick time-to-answer would be valuable for screening of individuals at remote locations, underserved clinics, mobile units-both military and civilian-schools, workplace settings, border crossings. airports and public events.

### SUMMARY

The invention provides a respiratory diagnostic platform including a sample and assay breathalyzer cartridge, which may be disposable, and a handheld analysis device (e.g . . . an actuator-detector-reader device for use with a breathalyzer cartridge) that can perform an analytical detection of respiratory pathogens captured from breath aerosols. During operation. the individual under test may expel breath through a removable or retractable blow tube connected to the disposable assay cartridge. A filter on the cartridge may capture breath aerosol droplets containing respiratory pathogens or free-floating respiratory pathogens. Next, the breath tube may be removed or retracted and the breath flow paths on the assay cartridge will be sealed. The assay cartridge may then be inserted into the handheld diagnostic device and a sequence of steps may occur. Initially, manual rupture or a motorized mechanism may rupture three blister packs containing aqueous-based buffers, pushing each into separate reservoirs subsequently used for sample preparation and isothermal amplification and detection. The cartridge also contains lyophilized reagents for purification of the pathogen nucleic acid and lyophilized reagents for the isothermal amplification with a fluorescent probe for detection by the handheld instrument. Once the detection of the fluorescent intensity and analysis is complete the self-contained cartridge can be safely and appropriately discarded.

### BRIEF DESCRIPTION OF THE DRAWINGS AND TABLES

FIG. 1. Overall embodiment of a respiratory breathalyzer detection system of the invention in which an individual being tested blows through a blow tube attached to the assay cartridge. A filter on the cartridge captures breath aerosols and particles that can harbor respiratory pathogens. The breath blow tube then may be removed or retracted from the cartridge and the cartridge may be inserted into the handheld instrument for running of the assay and analysis. A display on the instrument may provide results to the user.

FIG. 2. Shown is the expanded diagram of an assay cartridge illustrating the individual components. The top layer contains pressure/vacuum (pneumatic) mating ports for the manipulation of liquid during the running of the assay. Additionally, there are blister packs that contain the liquid reagents for the assay and an aperture for the imaging of the fluorescence of the reaction. The middle layer (fluidics layer) contains the fluidics channels, chambers and reservoirs, the excitation and emission window for the fluorescence and the breath filter. On the underside of the middle layer are safety moisture locks that prevent liquids from escaping the cartridge. The bottom layer contains waste liquids and houses an absorbent pad(s) or absorbent matrix. The layers are adhered with an adhesive film that is compatible with in vitro diagnostics, possessing a high transmission and low auto fluorescence across visible light wavelengths. The cartridge is designed without valves and is compatible with 3-dimensional printing and mold-injection manufacturing processes. In an embodiment shown, the middle layer inserts into a U-shaped clip to which a blow tube is attached, while the top and bottom layers about the two walls at the tip of the “U.” In an embodiment shown, the blow tube can be detachable. In a separate embodiment, the blow tube can be retractable.

FIG. 3. Shown is the assay cartridge fluidic layer: (A) The breath filter used for the capture of breath aerosols and particles. (B) The breath filter gasket used to secure the breath filter to the fluidics layer. (C) The breath filter and lysis chamber used for the lysis of captured pathogens. (D) The lysis buffer reservoir connected to (C) via an underside microchannel and used to store lysis buffer from the ruptured lysis buffer blister pack. (E) The wash buffer reservoir used to store wash buffer from the ruptured wash buffer blister pack. (F) The elution buffer reservoir used to store elution buffer from the ruptured elution buffer blister pack. (G) The magnetic beads and sample preparation chamber used for holding the magnetic beads and for the sample preparation process. (H) The reagent chamber holds lyophilized reagents for mixing with the elution buffer from the sample preparation. (I) The isothermal reaction chamber is where the isothermal reaction occurs. This chamber contains excitation and emission window(s) for fluorescence excitation and detection. The chamber can also contain lyophilized reagents and a stir bar for mixing reagents. (J) The pneumatic ports provide access for the pressure and vacuum system of the instrument for manipulating liquids in the fluidics. (K) The waste/vent/pneumatic ports provide access for the pressure and vacuum system of the instrument for manipulating liquids in the waste and fluidics layers and/or venting through the waste layer. Lighter lines show interior structure and microchannels of the manufactured fluidic layer.

FIG. 4. Overview image of the breadboard instrument showing the various subunits: (A) The instrument main controller and data processing section. (B) The fluorescent imager with optical components. (C) The pneumatic interface and valves to interface with the assay cartridge. (D) The magnetic manipulator used to hold and disperse the magnetic beads during the sample preparation process. (E) Electronic components for the control of the motors. valves and power conversion. (F) The pressure and vacuum source with a linear actuator. (G) Electronics for control of the imaging camera and the excitation source.

FIG. 5. CAD illustration of the assay cartridge holder of the breadboard instrument illustrating the magnetic manipulator, pneumatic interface, optical housings and heat-block and mixing motor compartment.

FIG. 6. CAD illustration of the magnetic manipulator used to maintain as well as disperse the magnetic beads in the magnetic bead chamber (see FIG. 3G). This version of the manipulator uses a motor to control the rail gear to position the upper magnet (top magnet) or lower magnet (bottom magnet) at a desired position above or below the magnetic bead/sample preparation chamber, respectively. At the full extension of the rail gear position, the armature with both the upper and lower magnets places the lower magnet beneath the chamber and the upper is moved away to transfer the magnetic beads to the bottom of the chamber. Retraction of the rail gear moves the lower magnet distal and the upper magnet proximal to transfer the magnetic beads to the top of the chamber.

FIG. 7. Shown is a graph depicting the evaluation results of multiple filter types for incorporation as the breath filter for the assay cartridge. The relative recovery was determined based on the placement of a quantified amount of live virus (influenza A) on to the various filters via pipet with a short incubation period (ca. 2 minutes) before elution from the filters using the lysis buffer of the sample preparation with the eluent processed using the sample preparation designed for the assay cartridge and quantified by real-time PCR. The flow rate was determined using a pump set to 8 L/min through a variable area flow meter (+/−4% accuracy) on a cross section area of 78.5 mm², which is the exposed area of the filter in the assay cartridge. Pore size or equivalent pore size is indicated after each filter type. Empirical assessment was used to determine a cut off for easy-of-operation by a healthy individual blowing across the individual filters. In general, any membrane, filter or matrix with sufficient flow rate for breath sampling and pore size or effective pore size to capture breath aerosols and particles and that is compatible with the sample preparation could be used.

FIG. 8. Fluorescence intensity versus time results for an automated assay run using the assay cartridge and the breadboard instrument. The RT-RPA-exonuclease probe reaction used 1e6 copies of SARS-CoV2 cDNA spiked onto the breath tilter of an assay cartridge before initiation of the automated run that included sample preparation and reaction monitoring and analysis. This proof-of-principle run at 42° C. required 35 minutes (12 minutes of sample preparation and ca. 23 minutes to the completion of the isothermal reaction).

FIG. 9. Fluorescence intensity versus time results for an alternative reaction using the T7-RT-RPA-molecular beacon probe method. This reaction was performed with 1000 copies of synthetic template and at an isothermal temperature of 39.5° C. on a laboratory real-time PCR thermal cycling instrument.

FIG. 10. Flow diagram of the all-in-one chamber RT-RPA-T7-MB process: (a) Reverse transcription of the RNA template; (b) eDNA product; (c) RPA (d) RPA amplification products that can be cycled back for re-amplification or carried forward; (e) T7 polymerase runoff using the primer-inserted T7 promoter site; (f) single-stranded RNA runoff product with bound molecular beacon (MB).

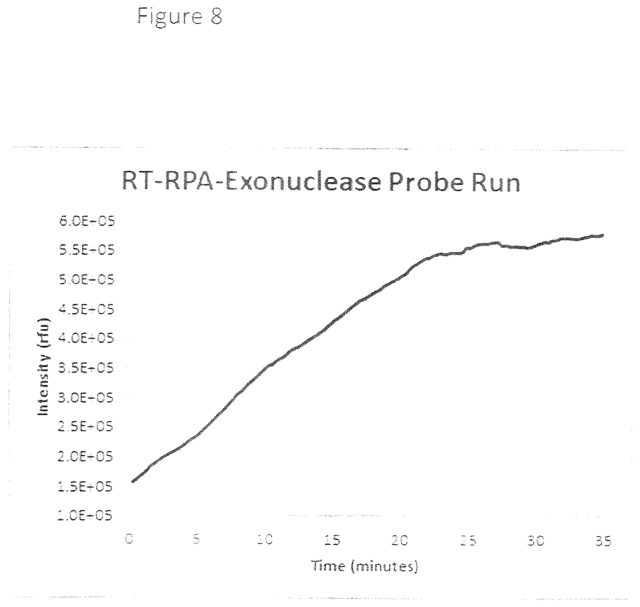

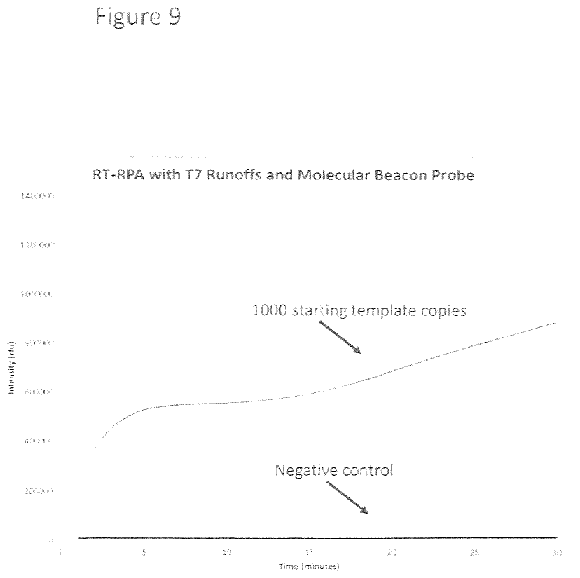

Table 1 (A & B). (A) List of primers and exonuclease probe targeting the nucleocapsid region of SARS-COV-2 for the RT-RPA-exonuclease probe reaction used in the automated run of FIG. 8. (B) List for primers, template and molecular beacon (6-carboxyfluorescein (6-FAM)/Black Hole Quencher®-1 (BHQ1) pair-labeled hairpin oligonucleotide) to test the RT-RPA-T7-Molecular Beacon reaction of FIG. 9.

### DETAILED DESCRIPTION OF THE INVENTION

Unless defined otherwise, all technical and scientific terms used herein have the same meaning as is commonly understood by one of ordinary skill in the art to which this invention belongs.

As used in the description of the invention and the appended claims, the singular forms “a”, “an” and “the” are used interchangeably and intended to include the plural forms as well and fall within each meaning, unless the context clearly indicates otherwise. Also, as used herein, “and” or “or” refers to and encompasses any and all possible combinations of one or more or two or more of the listed items, as well as the lack of combinations when interpreted in the alternative (“or”).

As used herein, “one or more” is intended to mean “at least one” or “all of the listed elements and a combination thereof”.

Except where noted otherwise, capitalized and non-capitalized forms of all terms fall within each meaning.

Unless otherwise indicated, it is to be understood that all numbers expressing quantities, ratios, and numerical properties of ingredients, reaction conditions, and so forth used in the specification and claims are contemplated to be able to be modified in all instances by the term “about” or “approximately”. As used herein, the term “about” when used before a numerical designation, e.g., temperature, time, amount, concentration, and such other. including a range, indicates approximations which may vary by (+) or (−) 10%, 5% or 1%.

The present and preferred embodiment of the invention is a sample and assay breathalyzer cartridge for breath sampling and performance of the assay along with a handheld device for providing a means to mechanically rupture blister packs, manipulate liquids and magnetic beads within the cartridge, capture fluorescence, perform analysis and provide an interface for the user. The cartridge may be disposable. The cartridge may be composed of multiple layers, preferably three, that can be manufactured under various processes. The first layer (upper layer) may contain blister packs that hold liquids required for the reaction and contain apertures for attachment of a blow tube or conduit to guide sample flow or entry. pressure and or vacuum ports to mate with an actuator-detector-reader device (which may be a handheld instrument) and can contain an exit vent for the fluid flow. The second or middle layer contains the fluidic paths that include reservoirs for the ruptured blister packs, chambers and channels, lyophilized reagents for sample preparation and the isothermal amplification reaction, optical windows for excitation and emission of the fluorescent probe/s and a filter membrane or matrix to capture breath respiratory aerosols, and can contain an exit vent pass through for fluid flow. The third or lower layer is for waste storage that contains an absorbent pad(s) or matrix and ports that lead to a moisture lock situated beneath the fluidics layer to maintain all liquids and reagents within the disposable cartridge. In an embodiment. the cartridge is valveless. In an embodiment, the cartridge comprises a valve. In an embodiment, the cartridge comprises a valve which is a one-way check valve. In an embodiment, the cartridge can be sealed so as not to allow liquid movement out of the cartridge.

Drawings with indicated dimensions provide an embodiment for the practice of the invention. It is to be understood that dimensions may be altered or the location of the relative placement of functional compartments or components may be altered without affecting the overall function or usability in other embodiments.

### Sample Preparation and Isothermal Reaction

Breath sample processing is based on lysis of the respiratory pathogens captured on the filter membrane using a lysis buffer at room or elevated temperature. The lysis buffer should be compatible (i.e., low pH or adjustment to low pH of less than pH 6.5) with charge dependent binding of nucleic acids to magnetic beads for sample preparation purposes. As such, the lysis buffer functions not only to release nucleic acid but is ready to support nucleic acid binding to magnetic beads, if not, may be modified post lysis, that is to a lower pH by the inclusion of a solid state organic acid (e.g., malie, citric, oxalic, etc.) in the magnetic bead chamber to ensure nucleic acid binding to the magnetic beads. After the nucleic acid of the respiratory pathogen binds to the magnetic beads, the beads are washed with a ph neutral to slightly acidic buffer (typically less than pH 7) to remove any contaminates that could interfere with the isothermal reaction. A high pH of greater than pH 8 buffer is used to elute the purified pathogen nucleic acid from the magnetic beads to be used as the genomic starting material for the isothermal reaction. A stir bar(s) in the reaction and/or reagent chamber(s) ensures mixing of reagents. The stir bar is controlled by a miniature motor equipped with a magnet or circuitry to produce a rotating magnetic field. The motor is placed directly beneath the reaction chamber and when activated spins the stir har as directed by the controlling software/firmware. This step can also be used anywhere in the fluidic pathways that require mixing of liquids.

10025| The isothermal reaction can be one of several: recombinase polymerase amplification (RPA), loop mediated amplification (LAMP), helicase dependent amplification (HDA), or any nucleic acid amplification method performed at a single temperature without a need for thermal cycling between amplifications. Nucleic acid may be R.NA or DNA. In an embodiment. RNA may be converted to DNA through the use of a reverse transcriptase prior to amplification, wherein amplification is amplification of the cDNA. In an embodiment, the reverse transcriptase may possess RNase H activity. In a separate embodiment, the reverse transcriptase may lack RNase H activity. In an embodiment, reverse transcription may be carried out in the presence of a single-strand-binding protein (ssb). In an embodiment, eDNA is amplified under conditions permissive for activity of a DNA polymerase in the presence of ssb. In an embodiment, amplification is carried out on isolated nucleic acid in a single reagent mix comprising primers, enzymes, buffer, salts. The single reagent mix may additionally comprise a detection system to monitor or quantify resulting amplified nucleic acids.

In one embodiment, the isothermal reaction may use reverse transcriptase in combination with recombinase polymerase amplification and an exonuclease probe (RT-RPA-exonuclease probe) as a single mix in an R.T-R.PA-exonuclease-exonuclease probe molecular assay. For RPA, the primers are designed to target regions of the genome for pathogen identification. For RNA viruses or other RNA targets, the reverse transcriptase replicates the template as cDNA prior to the recombinase enzyme implementing a strand exchange by annealing the primers to the targeted sequence. For DNA viruses or DNA target the reverse transcription is not required. Once the primers bind, the polymerase amplifies while displacing the complement strand. The newly amplified sequence is cycled back to repeat this process with further amplification of the cDNA in the RPA procedure. To detect or quantify presence of a target sequence in the amplified cDNA. an exonuclease probe is present in the single mix. The exonuclease probe comprises a complementary DNA sequence to a target sequence in the amplified nucleic acid. The exonuclease probe further comprises an abasic site (i.e., apurinic/apyrimidinic site) between a fluorophore and a quenching moiety wherein the quenching moiety effectively quenches fluorescence of the fluorophore. The exonuclease probe additionally comprises a polymerase blocking agent at the 3′ end (e.g., a C3 spacer, denoted as 3-Sp3 in Table 1). During the course of the RPA amplification, the exonuclease probe may hybridize to its target sequence in the cDNA and be cleaved at an abasic site by apurinic/apyrimidinic endonuclease activity of an exonuclease enzyme (e.g., Exonuclease (I) that permits spatial separation of the fluorophore and the quenching moiety. This separation of the fluorophore from its quenching moiety causes an increase in fluorescence, directly related to amount of RPA product. The overall process can be extremely fast, yielding observable results in S to 10 minutes, depending on the nature of the nucleic acid (e.g., RNA or DNA, location of primer binding site and sequence, etc.) and starting copies of the targeted pathogen.

In a separate embodiment, the isothermal reaction can use a modified version of RPA that is designed as a singular reaction mixture of RPA reagents, reverse transcriptase (RT), T7 polymerase (T7) and a target specific molecular beacon (MB). In such embodiment, the single reaction mixture comprises (1) RPA reagents comprising a primer with at least one comprising a T7 RNA polymerase promoter sequence, DNA polymerase, divalent metal, salt and buffer and (2) T7 RNA polymerase. In another embodiment of the invention, the single reaction mixture comprises a bacteriophage RNA polymerase and a promoter sequence corresponding to the bacteriophage RNA polymerase other than T7 RNA polymerase and T7 RNA polymerase promoter sequences in the primer used for isothermal amplification. Other . . . bacteriophage RNA polymerases and promoter sequences include but are not restricted to SP6 RNA polymerase and its promoter sequences and T3 RNA polymerase and its promoter sequences.

In an embodiment, this isothermal, RT-RPA-T7-MB molecular assay may combine all reaction components in a single mix. For RPA, the primers are designed to target regions of the genome for pathogen identification with one primer comprising a bacteriophage RNA polymerase promoter sequence. For RNA viruses or other RNA targets, the reverse transcriptase replicates the template as eDNA prior to the recombinase enzyme implementing a strand exchange by annealing the primers to the targeted sequence. Once the primers bind. the polymerase (DNA polymerase) amplifies while displacing the complement strand. The newly amplified sequence is cycled back to repeat this process. In this assay, one of the primers contains the T7 promoter site on the S′ end, which permits the T7 RNA polymerase to run off a single-stranded complement sequence of RNA. Importantly, the molecular beacon or alternative probe (e.g., linear fluorescent probe with competitive quencher fragment) is specific to this region between the primers. Thus, only correctly targeted amplification results in detection—this mitigates nonspecific positive results typically associated with some types of isothermal reactions. The overall process can be extremely fast, yielding observable results in S to 10 minutes, depending on the nature of the nucleic acid and the starting copies of the targeted pathogen.

### Hardware

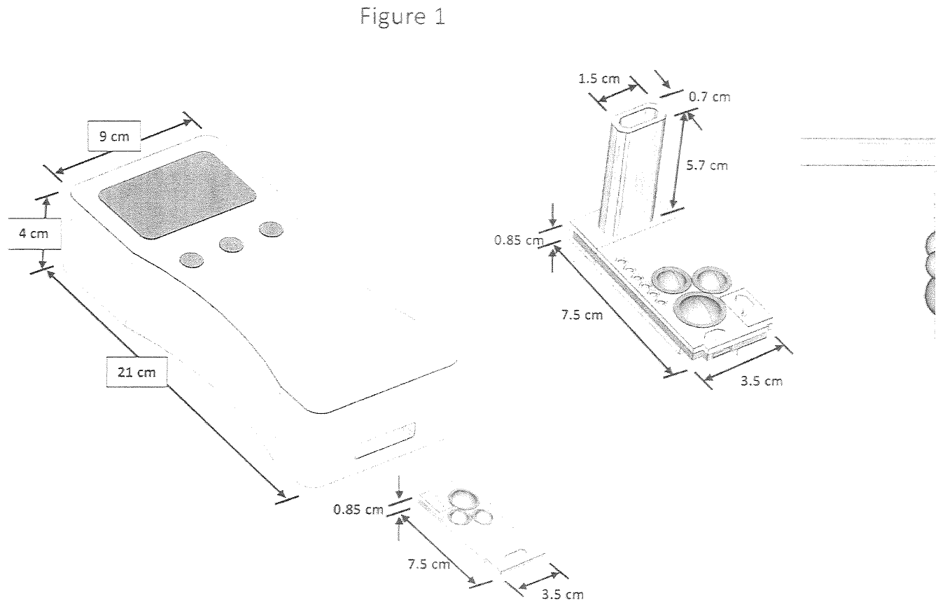

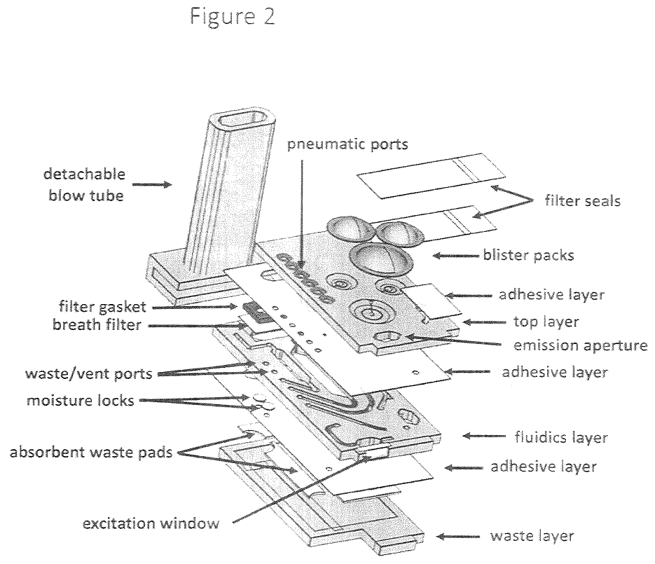

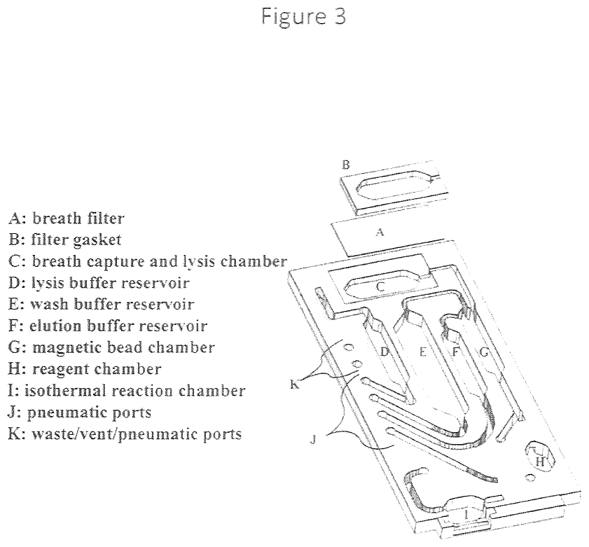

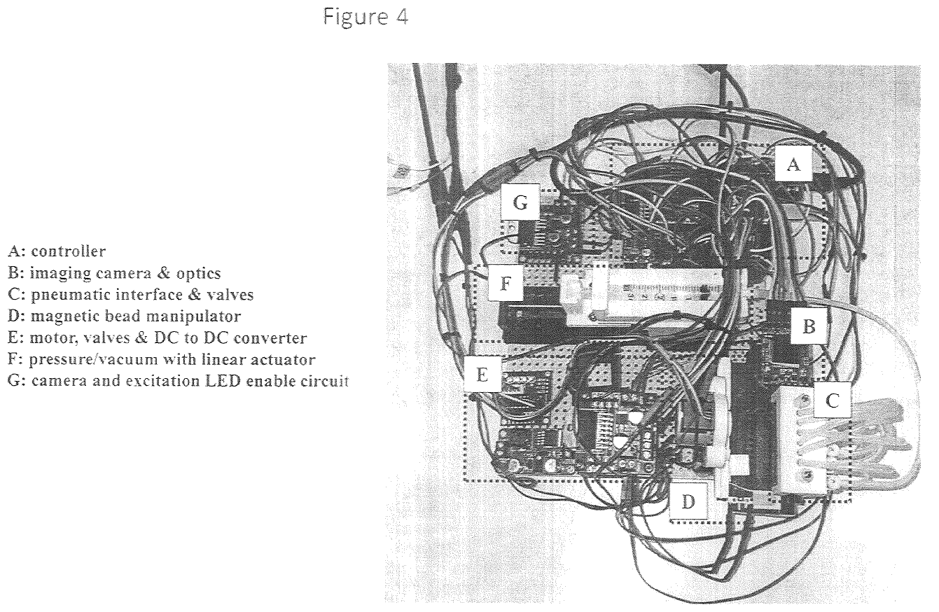

During operation, the individual under test may blow through a blow tube fitted to the disposable assay cartridge, FIGS. 1 and 2. The exhaled breath goes through the blow tube and across a membrane, breath filter, as shown in FIGS. 2 and 3, that captures breath aerosols and particles on the filter. When the breath sample has been collected, the blow tube is removed or retracted, and the cartridge entrance and exit to the filter are sealed, which can be accomplished by various means, such as sliding clips, insertion of plugs, sealing tape, or similar ways and means of sealing entrance and exit to a cartridge. After sealing the filter, the cartridge is inserted into the handheld instrument (breadboard instrument version illustrated in FIG. 4), locking the cartridge into place while mating the instrument pressure/vacuum capabilities to the cartridge pressure/vacuum ports. If only pressure or vacuum is to be employed a one-way check valve can be included to limit liquid movement to one direction.

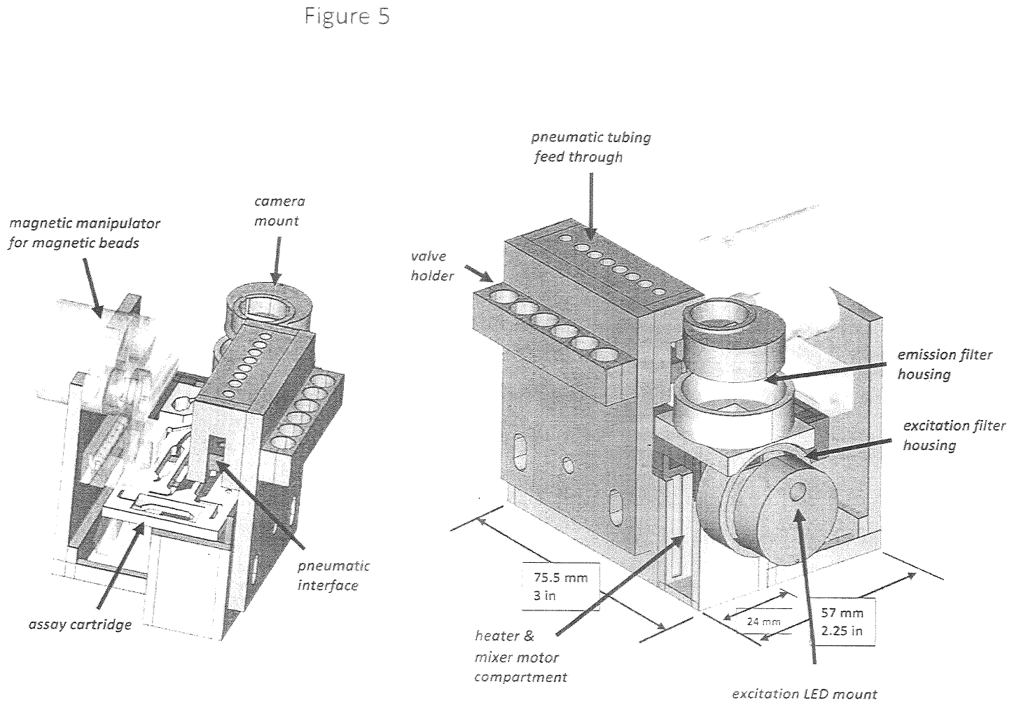

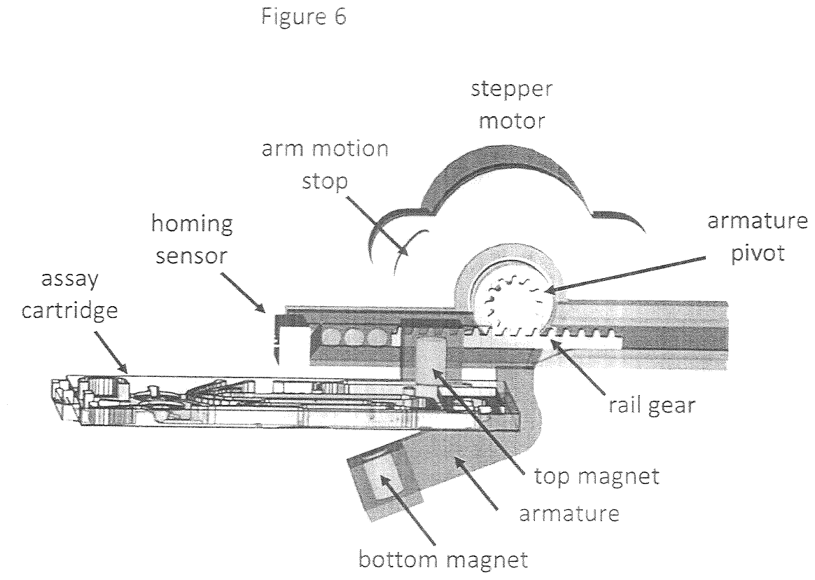

Once inserted and locked, the instrument runs a motorized car that ruptures the blister packs on the cartridge (FIG. 2), sending the liquids into respective holding reservoirs (FIGS. 1, 2 and 3). Using the pressure/vacuum port (FIG. 3J) associated with the lysis buffer (FIG. 3D)), the instrument applies pressure or vacuum to push or pull the buffer onto the filter (FIG. 3A) in the breath capture and lysis chamber (FIG. 3C), where it resides for a specified time to lyse and release any pathogens captured on the filter. In the embodiment shown, pressure is used to push the lysis buffer onto the filter in the breath capture and lysis chamber or pull the lysis buffer by vacuum applied through the waste chamber using the waste/vent port (FIGS. 3C and 3K). Alternatively, by changing the microfluidic pathway, location of the blister packs or location of the blister pack/reservoir. the instrument may uses other combinations of pressure and vacuum to push or pull the buffer onto the filter in the breath capture and lysis chamber. After lysis, the lysis buffer, with lysed pathogens, is pushed or pulled through a microfluidie channel to the sample preparation magnetic bead chamber (FIG. 30) where it rehydrates the magnetic purification beads and binding reagents. The magnetic bead manipulator (depicted in FIGS. 5 and 6) within the instrument can be used to mix the magnetic beads with the lysis buffer. In an embodiment, mixing may be achieved by cycling the relative position of the top and bottom magnets (FIG. 6) in relation to the magnetic bead chamber (FIG. 30). At this point and after an incubation time, the lysed-pathogen nucleic acid adsorbs to the magnetic beads and the magnetic bead manipulator can then secure the magnetic beads to a side of the chamber. When the beads are secured, the lysis buffer can be pushed/pull, by pressure/vacuum, out of the chamber and into waste (FIG. 2) via a microfluidic channel or returned to the breath filter/lysis chamber (FIG. 3C). Once the lysis buffer is secured, the pressure or vacuum is applied to move the wash buffer from its reservoir (FIG. 3E) into the sample preparation chamber (FIG. 3G) to remove any contaminants. At this point the magnet manipulator can disperse the magnetic beads to facilitate washing of the beads, after which it can re-secure the beads. After sufficient washing or wash cycles, the wash buffer is pushed or pulled and secured into the waste layer (FIG. 2) or the wash buffer reservoir (FIG. 3E). Pressure or vacuum is then used to move the elution buffer in its reservoir (FIG. 3F) into the magnetic bead chamber (FIG. 3G). In the embodiment shown in FIGS. 3E and 3F, pressure is used to push the wash buffer and elution buffer from their respective reservoirs. However, in a separate embodiment, vacuum may be used to pull the wash buffer or elution buffer from its respective reservoir into the magnetic bead chamber by changing the microfluidic pathway, location of the blister packs or location of the blister pack/reservoir. During incubation, the purified pathogen nucleic acid is released into the buffer--the magnet manipulator can be used to disperse the beads to facilitate the efficiency of elution. After the magnetic heads are secured in the chamber by the magnet manipulator (FIGS. 5 and 6), the elation buffer is pushed or pulled into the reagent chamber (FIG. 3H) to rehydrate and mix with the isothermal reaction reagents and then moved into the reaction chamber (FIG. 31). In the embodiment shown in FIGS. 3E and 3F, pressure is used to push the elution buffer into the reagent chamber (FIG. 3H). fo a separate embodiment, vacuum may be used to pull the elution buffer from the magnetic bead chamber to the reagent chamber by changing the microfluidic pathway or location of the magnetic bead chamber to the reagent chamber. As an alternative. the elution buffer can proceed directly to the reaction chamber if the isothermal reagents are stored there. The reaction matrix is then heated to the preferred temperature of the isothermal reaction—preheating the chamber can also be accomplished. The beating can be accomplished by a beater in the handheld instrument that is physically in contact with the reaction chamber, a small heating element incorporated into the cartridge or other appropriate means As the reaction progresses, the instrument's excitation source illuminates the reaction chamber and the generated fluorescence is captured by the detector of the instrument, preferably near or at 90º degrees to the excitation. Fluorescence emission is measured at set time points for analysis. The instrument, after data analysis, displays the results as either a positive, negative or invalid.

The instrument may be hand held and may contain a docking port for the cartridge. The hand held has a pressure/vacuum source and manifold valves to direct pressure or vacuum to the appropriated pressure/vacuum ports on the cartridge. The hand held also contains an illumination source and detector with respective optical filters for excitation and detection of the fluorescence associated with the fluorophore used. A heater, stir bar motor and moveable magnets of a magnetic bead manipulator can also be incorporated in to the instrument. The sequence of events as well as data analysis is controlled by a microprocessor housed in the handheld. Data analysis is accomplished by an algorithm designed to analyze the acquired fluorescent intensity versus time data resulting from the progression of the isothermal reaction. The analysis software uses the output data to determine the minimum and maximum start position of the curve, the maximum curve end position and the length of the minimum and maximum curve length. The algorithm seans across the curve in segments generating fits to a 2nd order polynomial or other order polynomial. Output of the analysis generates an eight-member array or other member array that includes: polynomial equation (ax² +bx+c) with terms a (curvature), b (slope), e (baseline offset) and R² (regression fit), the start, the end, the search maximized score and the curve-weighted score. Positive reactions have a greater positive slope parameter and a negative curvature parameter when nearing reaction completion while negative reactions possess a less positive slope parameter and a curvature that is near zero or positive for the observed time period of the reaction. All windows between minimum and maximum length over the minimum and maximum range are evaluated. A search score is generated: Search score= [b * R²*(−1a) * (% of range covered)]. A weight score is then determined: Weighted score= [search score * (—1a)]. The symbol “*” is a multiplication symbol. The weighted score is not implemented for the search as it increases the occurrence of short window local minima with sharper slopes and a short negative inflection, which would be less sensitive for differentiating negatives from low-input positives. The positive to negative threshold of the weighted score is determined by characterization of empirical sets of known positive and negative data. Implementation of an internal positive control using a complementary fluorescent probe and expanded handheld capabilities for dual detection could also be added to aid in positive, negative and invalid results.

Results are displayed on a liquid crystal display or similar item. Alternatively, or additionally, sound may be used to report on results.

Advantages of the invention include methods, cartridges and devices (e.g., handheld devices) that provide the ability to detect respiratory pathogens from breath aerosols using pathogen nucleic acids, fluorescent probes and sensitive isothermal amplification. Further, the present embodiment of devices, cartridges and methods of the invention provides for a molecular amplification assay in the field (i.e., point-of-care) with an approximate 30-to-40 minute detection period. Merely as an example, point-of-care identification of a respiratory pathogen may be accomplished at doctor's offices, remote medical clinics, work sites, airports, public events, schools, or military front-line medical clinics. Additionally, the device and methods of the invention may be configured-manufactured-for alternate respiratory pathogens. Moreover, the cartridges are self-contained and disposable.

The following example is presented to illustrate the present invention and to assist one of ordinary skill in making and using the same. The example is not intended in any way to otherwise limit the scope of the invention.

### Example I

Assay Description (FIG. 1): The assay is developed to detect respiratory pathogens after an individual blows through a blow tube that is connected to the cartridge. The blow tube allows the breath of the individual, at high relative humidity, to pass through a filter membrane or matrix built into the assay cartridge. After collection, the blow tube is removed or retracted, depending on design, and the breath entrance and exit of the filter is sealed prior to insertion into the instrument. The assay process involves lysing any respiratory pathogens collected on the filter such that the genomic nucleic acids of the pathogen/s are released into the lysis buffer. The lysis buffer is then mixed with a charge-based purification system involving magnetic beads that can adsorb the nucleic acid for purification from contaminates that potentially would inhibit the subsequent amplification reaction. After purification, the nucleic acids are introduced into an isotherm reaction matrix where amplification reaction primers are specifically designed for the pathogen of interest. If the pathogen of interest is present, the isothermal amplification proceeds by amplifying the genomic target of interest and a fluorescent probe interacts with the target to fluoresce at an appropriate excitation wavelength. The fluorescence is recorded and its intensity readings are used to determine the presence of absence of the respiratory pathogen. The handheld instrument automatically performs the manipulation of the liquids within the cartridge, magnetic dispersion and sequestering of the magnetic beads, the isothermal reaction and imaging of the fluorescence and analysis. A user display on the handheld instrument displays to the user the results of the testing.

Assay Cartridge Design (FIG. 2): The assay cartridge, in the form presented, is constructed of three layers: top layer, fluidics layer, waste layer. The top layer provides a base for the three liquid blister packs. The largest blister pack is for the wash buffer, the others are for the lysis buffer and elution buffer. When mechanically ruptured by mechanical pressure supplied by the instrument and with the aid of a sharp projectile manufactured into the top layer yet beneath each of the blister packs, the liquid flows through the top layer and into the fluidics layer. The top layer also contains an opening or cut away for the breath filter--where the blow tube mates--and pressure/vacuum ports that mate with the instruments pressure/vacuum system. An opening exists for the emission window above the reaction chamber for measuring fluorescent intensities.

The fluidics layer houses the microfluidic channels, chambers, buffer reservoirs, lyophilized magnetic beads and lyophilized binding agents in the magnetic bead chamber. lyophilized isothermal reaction reagents in the reagent and/or reaction chamber, a stir bar in the isothermal reaction chamber and/or reagent chamber, waste channels for pushing or pulling expended buffers to waste, breath filter aperture and moisture locks to prevent liquid from escaping the cartridge or reaching the pneumatics of the instrument. In addition, the fluidics layer comprises one or more optical window(s) for fluorescence excitation and emission.

The waste layer is for storing discarded buffers and contains an absorbent pad(s) or matrix that secures the waste to prevent leakage. The waste layer may contain multiple chambers for alternative pathways for waste liquids.

Microfluidic layer and process (FIG. 3): In one example, the microfluidic layer of the cartridge may contain the membrane filter, that is used to collect the breath aerosols and particulates when the person under test blows through the blow tube attached to the assay cartridge. During operation, the blister packs are ruptured and dispense the individual liquids into the respective reservoirs for the sample preparation process. Reservoir (D) is for the lysis buffer. reservoir (E) is for the wash buffer and reservoir (F) is for the elution buffer. The sample preparation/magnetic bead chamber. (G), comprises nucleic acid purification magnetic beads and binding reagents. The binding reagents and magnetic beads may be lyophilized for extended shelf-life. In the isotheral reaction chamber, (I), are lyophilized isothermal reaction reagents that include at least one fluorescent probe for detection of the progression of the reaction. An optional stir bar can be included in the isothermal reaction chamber to facilitate mixing. (H) the reagent chamber can also contain lyophilized reagents for the isothermal reaction with an additional stir bar, if premixing is desired. After the blow tube is removed, the filter entrance and exit may be sealed with air and liquid secure fit plugs or membranes, and the assay cartridge may be inserted into the instrument. The blister packs are mechanically ruptured by the instrument and the liquid disperses down through the top layer and enters the appropriate reservoirs. Metered pressure or vacuum from the instrument through the (J) port associated with the lysis buffer reservoir pushes the lysis buffer to the filter membrane, entering above or below the membrane and encompassing the membrane in the lysis buffer. After sufficient incubation time, during which the respiratory pathogens are lysed to release the associated genomie nucleic acids, the lysis buffer is moved forward through the microfluidic channel to the sample preparation/magnetic bead chamber. (0). In the sample preparation/magnetic bead chamber, the lysis buffer rehydrates the binding reagent(s) and the magnetic beads, after which the magnetic beads are dispersed throughout the lysis buffer by the magnetic bead manipulator. The rehydrated mixture is permitted to incubate as the nucleic acid from the lysed respiratory pathogens binds to the surface of the magnetic beads. When the incubation has concluded, a magnet bead manipulator can sequester the magnetic beads as the liquid lysis buffer is pushed or pulled to the waste layer or returned to the breath filter and lysis chamber (C). Next using the port (J) associated with the wash buffer, pressure or vacuum is applied to slowly push or pull the wash buffer from the wash reservoir (E) to the magnetic bead chamber, respectively. The magnet can remain in place with slow passage of the wash buffer over the sequestered magnetic beads to wash away any contaminates. leaving the nucleic acid bound to the beads. The used wash buffer is then pushed or pulled to waste through the waste channel prior to the reagent chamber using metered pressure of vacuum through the associated wash buffer port (J). Optionally, the wash buffer can be moved in increments, which during each increment the magnetic bead manipulator is used to mix the buffer and beads (potentially by pressure/vacuum, magnet movement or etc.) then re-collected by bringing the magnet proximal to the mixing chamber before pushing the wash buffer to waste. The final buffer is the elation buffer, which using pressure or vacuum from the associated pressure port (J), is moved to the magnetic bead chamber with the magnetic beads. The beads are incubated with the elution buffer to elute the bound nucleic acids off the magnetic beads. Again here. the magnets can be moved distal and proximal to the mixing chamber to disperse the beads for more efficient elution prior to re-collection of the beads. Once the nucleic acid is eloted, the eluate is pushed or pulled to the reagent and reaction chambers (H & 1) and rehydrate the lyophilized isothermal reaction components in the chamber/s. Ports (K) can be used with pressure or vacuum to manipulated liquids in the waste and fluidics layer or be used as vents for the fluidics layer. The buffer with reaction components remains in the reaction chamber as the reaction proceeds at an isothermal temperature and a stir bar is used for mixing of the reagent and aid in reaction efficiency. As the reaction proceeds, excitation and emission of the fluorescent probe/s detail the progression of the isothermal reaction to determine the presence or absence of respiratory pathogen nucleic acids. Multiplex detection can also be accomplished using multiple fluorophores.

Instrument Design (FIGS. 4, 5 and 6): The seven basic subsections of the breadboard version of the handheld instrument are illustrated in FIG. 4. A though O. Subsection λ is the controller that contains the control software to control operation and interaction of the subsections and the analysis software. The imaging camera and optics are associated with subsection B with holders and housing observable in FIG. 5. For the breadboard the excitation is accomplished with a white light emitting diode with excitation wavelength limited by a fluorophore specific bandpass filter. The emission from the fluorescent probe(s) pass through a fluorophore specific bandpass filter before capture by the imaging detector (e.g . . . a CMOS or CCD camera chip). The pneumatic interface and valves, subsection C couples the pressure/vacuum source to the assay cartridge. The magnetic bead manipulator, which is implemented to control the magnetic beads within the cartridge, is subsection D and also shown in depth in FIG. 6. The magnetic bead manipulator uses a motor driven gearing system and magnets to mix or sequester the magnetic beads in the magnetic bead chamber of the assay cartridge. Pressure and vacuum is generated in subsection F that is a linear actuator with a syringe; other options could include pressure and vacuum pumps, flexible chambers that are compressed or expanded by actuators or alternative means. Subsection E contains the electronic circuits that control the valves and motors and a step down direct current to direct current converter. Additional electronics are in subsection G to control the camera and enable the excitation LED

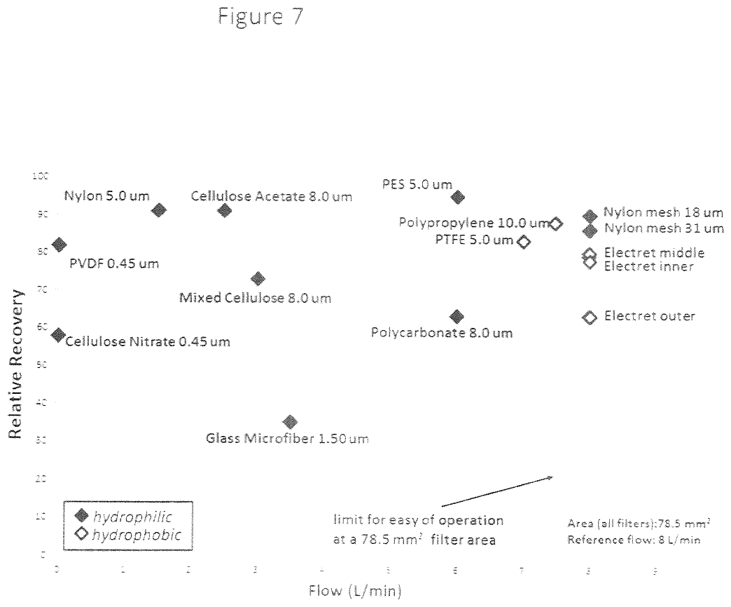

Breath filter Selection for the Assay Cartridge (FIG. 7): The breath filter is any membrane or filter that is assay cartridge compatible and capable of capturing the breath aerosols with sufficient flow that an individual can blow across the filter without difficulty or excessive difficulty. FIG. 7 depicts the test results of several commercially available materials. A known amount of live viral particles (influenza A) as a liquid suspension is deposited via pipet onto the various test filters with a short incubation period (ca. 2 minutes) before elution from the filters using the lysis buffer of the sample preparation with the eluent processed using the sample preparation designed for the assay cartridge and quantified by real-time PCR. The graph shows relative recovery of viral nucleic acid from the test filter in relation to no filter control where the live viral particles are directly mixed with the sample preparation lysis buffer. Flow rates were determined using a variable area flow meter to measure air flow across an area of 78.5 mm² for each of the filter materials using a no filter flow of 8.0 L/min. Inclusion for cartridge testing was limited to high flow rates and high viral recovery. The qualitative cut off of 7.5 L/min for flow rate was selected, as any filter with flow below this level proved difficult for an individual to blow across. In an embodiment, suitable filter for the assay cartridge is selected from the group consisting of polypropylene, nylon mesh, electret and filters providing greater than 60% recovery of a respiratory pathogen and supporting about 7.5 L/min or greater flow rate when subjected to about 8 L/min no filter reference flow rate for a 78.5 mm² filter area.

RT-RPA-Exonuclease Probe Results (FIG. 8): Shown is the data of an RT-RPA-exonuclease probe assay from an automated run using the laboratory breadboard instrument and assay cartridge with a spike of 1e6 copies of SARS-COV-2 cDNA pipetted on the breath filter of the assay cartridge before the cartridge was sealed and placed into the bread board instrument for the automated run. The reaction was run at 42° C. for 40 minutes. Total sample preparation time was 12 minutes. Data points were acquired at 16 second intervals, The automated data analysis was able to correctly identify the positive sample with a weighted score of 22.545.

RT-RPA-T7-Molecular Beacon Probe Results (FIG. 9), Shown is an alternative isothermal assay using RT-RPA-T7-molecular beacon reaction. The reaction was run as a 25 ul, reaction volume at 39.5° C. for 30 minutes. Fluorescent intensity levels were captured at 1-minute intervals. The starting template was 1000 copies of the synthetic template per 25 ul. reaction. The negative control contained the same reaction components except no template was included. The reaction was run on a real-time PCR instrument operating in an isothermal mode.

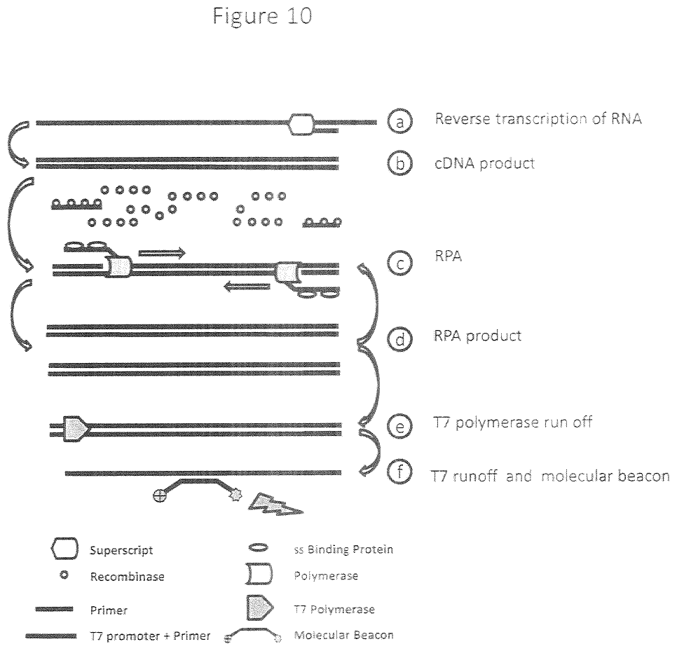

Reaction Process (FIG. 10): The schematic illustrates the principle of RT-RPA-T7 with a molecular beacon probe assay in an isothermal reaction using recombinase polymerase amplification (RPA) with the addition of reverse transcriptase (RT), T7 RNA polymerase (T7). and a molecular beacon or alternative probe (e.g., linear fluorescent probe with competitive quencher fragment). During RPA. the primers are specifically targeted to a bioinformatically significant region of the pathogen for selective identification. For RNA viruses or other RNA targets, the reverse transcriptase reverse transcribes the RNA template to produce Ist strand cDNA (step a), followed by 2nd strand synthesis by the polymerase (DNA polymerase) (step b). The double stranded DNA serves as a template for the recombinase enzyme to perform a strand exchange reaction resulting in annealing of the primers used in reverse transcription and 2nd strand cDNA synthesis to the targeted sequences (step c). Once each primer binds to respective DNA template, the DNA polymerase extends the primers while displacing the complement strand (step e). The newly amplified sequence is cycled back to repeat this process (steps c-d). As one of the primers contains a T7 promoter sequence on the S′ end, T7 RNA polymerase in the reaction binds to its promoter sequence in double stranded DNA (step e) and transcribes the downstream sequences to produce a single-stranded complement sequence of RNA (step f). The molecular beacon or alternative probe (e.g., linear fluorescent probe with competitive quencher fragment) is specific to this region between the primers, thus only correctly targeted amplification results in a detection—this mitigates nonspecific positive results typically associated with some types of isothermal reactions. The ability to perform a T7 RNA runoff transcription also permits probe binding without significant complementary strand competition, Once the probe is bound, fluorescent absorption and emission can oeeur for detection by the handheld instrument. The overall process can be extremely fast, yielding observable results in S to 10 minutes, depending on the nature of the nucleic acid and the number of starting copies of target pathogen. Note that in other embodiments, other bacteriophage promoter and bacteriophage RNA polymerase combination such as SP6 promoter and SP6 RNA polymerase, T3 promoter and T3 RNA polymerase, etc. may be used in place of T7 promoter and T7 RNA polymerase.

All components of the isothermal reaction can be combined as a single mixture: RPA reaction mixture with the addition of the T7 RNA polymerase, primers where one contains the T7 promoter sequence and a molecular beacon probe. For inclusion in the assay cartridge the RPA reagents including the T7 RNA polymerase, primers and probe can be lyophilized for rehydration after completion of the sample preparation, which could also contain lyophilized version of the magnetic heads and solid state acid as a binding facilitator. The liquid buffers would be contained in rupturable packs (blister packs) on the cartridge. The lysis buffer (e.g., detergent or surfactant based), wash buffer (neutral ph aqueous) and elution (elevated pH aqueous) are contained in blister packages of sufficient volume (e.g., lysis buffer ≥250 al . . . wash buffer >500 ul and elution buffer up to 200 uL) to accomplish sample preparation and rehydration of the RPA reagents.

For the RT-RPA-exonuclease and exonuclease probe molecular assay, the reverse transcription of viral RNA or other RNA target may occur as described for (step a) in FIG. 10, followed by cDNA synthesis (step b) and recombinase polymerase amplification (RPA) (steps c-d). However, steps (e) and (f) may be omitted. as transcription of the amplified DNA template is not required in the exonuclease molecular probe assay. As such, the primers used for amplification do not require a promoter sequence for a bacteriophage RNA polymerase (e.g . . . . T7 RNA promoter sequence) nor a bacteriophage RNA polymerase (e.g., T7 RNA polymerase) in the reaction mix. Instead, the reaction mix additionally comprises an exonuclease probe comprising an abasic site (i.e., apurinic/apyrimidinic site) between a fluorophore and a quenching moiety and an exonuclease (e.g., exonuclease III). The quenching moiety effectively quenches fluorescence of the fluorophore. The exonuclease probe may have a polymerase blocking agent at the 3′ end (e.g., a C3 spacer, denoted as 3-Sp3 in Table 1). During the course of the RPA amplification (step c), such as, for example, in the newly synthesized strand, the exonuclease probe may hybridize to its target sequence in the cDNA. and be cleaved at an abasic site by apurinic/apyrimidinie endonuclease activity of an exonuclease enzyme (e.g., Exonuclease III) that permits spatial separation of the fluorophore and the quenching moiety. This separation of the fluorophore from its quenching moiety causes an increase in fluorescence, directly related to amount of RPA product. The overall process can be extremely fast, yielding observable results in 5 to 10 minutes, depending on the nature of the nucleic acid and the starting copies of target pathogen.

All components can be combined as a single mixture: RPA reaction mixture with the addition of the exonuclease, primers and exonuclease probe. For inclusion in the assay cartridge the RPA reagents including the exonuclease, primers and exonuclease probe can be lyophilized for rehydration after completion of the sample preparation, which could also contain lyophilized version of the magnetic beads and solid state acid as a binding facilitator. The liquid buffers would be contained in rupturable packs (blister packs) on the cartridge. The lysis buller (e.g., detergent or surfactant based), wash buffer (neutral pHl aqueous) and elution (elevated pH aqueous) are contained in blister packages of sufficient volume (e.g., lysis buffer: : 250 μL, wash buffer ≥500 ul. and elution buffer up to 200 μL) to accomplish sample preparation and rehydration of the RPA reagents.

For data analysis, the detection algorithm scans across the curve in segments generating fits to a 2nd order polynomial or other order polynomial. Output of the analysis generates an eight member array or other member array that includes: polynomial equation (ax² +bx+c) with terms a (curvature), b (slope), c (baseline offset) and R² (regression fit), the start, the end, the search maximized score and the curve-weighted score. Positive reactions have a greater positive slope parameter and a negative curvature parameter when nearing reaction completion while negative reactions possess a less positive slope parameter and a curvature that is near zero or positive. During the reaction time period all windows between minimum and maximum length over the minimum and maximum range are evaluated. A search score is generated: Search score= [b * R²*(-1a) * (% of range covered)]. A weight score is then determined: Weighted score= [search score * (−1a). The symbol “*” is a multiplication symbol. The weighted score is not implemented for the search as it increases the occurrence of short window local minima with sharper slopes and a short negative inflection, which would be less sensitive for differentiating negatives from low-input positives. The positive to negative threshold is determined by characterization of empirical sets of known positive and negative data. Implementation of an internal positive control using a complementary fluorescent probe and expanded handheld capabilities for dual detection could also be added to aid in positive, negative and invalid results.

An example timeline of the overall process for the bread board: Sample collection is estimated to take up to two minutes for sample collection, cartridge sealing of breath filter, insertion into the instrument and initiating the assay run. Timed runs on the breadboard for the sample preparation portion of the assay are accomplished in 12 minutes. The RPA reaction is set to run for 40 minutes for testing and development to observe the reaction completion, though shorter times are envisioned. Data analysis is less than 15 second upon the conclusion of the reaction period. The overall time is less than 53 minutes.

Cartridge reagents can include: RPA reaction mixture: T7 RNA Polymerase or substitute; primers; probe or probes; exonuclease enzyme: Lysis Buffer detergent or surfactant buffer that can inchide the addition of proteinase K and is pH adjustable to a pH<6.5 to enable binding to the magnetic purification beads; Wash Buffer--buffered aqueous buffer of pH<6.5; Elution Buffer--aqueous buffer that may contain tris-HCl that is pH adjusted to >pH 8.0; Solid State Acid—acid in the solid or lyophilized form that can be malic acid or citric acid, ete. used to pH adjust the lysis buffer for binding to the magnetic purification beads.

The cartridge, point-of-care or handheld breathalyzer system, and methods of the invention can be used to detect respiratory pathogen(s) in breath or respiratory aerosol of a subject. The sample and assay breathalyzer cartridge, point-of-care or handheld breathalyzer system, and methods of the invention for detection of a respiratory pathogen(s) in the breath or respiratory aerosol of a subject can comprise the sample and assay breathalyzer cartridge of the invention and forward and reverse primers that target a nucleic acid sequence unique to the pathogen or shared by a set of said pathogens. The respiratory pathogen(s) can be a bacterium and/or a virus. The bacterium and/or virus can be viable, intact or infectious. Alternatively, the bacterium and/or virus can be not viable or not intact or exudes its genomic nucleic acid. The cartridge, point-of-care or handheld breathalyzer system. and methods of the invention can capture viable, intact or infectious bacterium or virus particle. The cartridge, point-of-care or handheld breathalyzer system, and methods of the invention can also capture bacterium and/or virus which is not viable or not intact or exudes its genomic nucleic acid. The genomic nucleic acid may be RNA or DNA.

In an embodiment, the respiratory pathogen(s) is a bacterium. Bacterial respiratory pathogens are known in the art and may be, but are not limited to, Streptococcus pneumoniae. Staphylococcus aureus. Methicillin-resistant Staphylococcus aureus (MRSA). Escherichia coli, Klebsiella pneumoniae, Pseudomonas aeruginosa. Acinetobacter baumannii, Stenotrophomonas maltophilia, Haemophilus influenzae, Legionella pneumophila. Mycoplasma Pneumonia, Chlamydia pneumonia. and Mycobacterium tuberculosis or any bacterium that may be present in a breath or respiratory aerosol of a subject. In a separate embodiment, the respiratory pathogen(s) is a virus. Viral respiratory pathogens include, but are not limited to, coronavirus, influenza virus, para influenza virus, rhinovirus (RV), measles virus, respiratory syncytial virus (RSV), human metapneumovirus (HMPV). buman bocavirus (HBoV) and any virus that may be present in a breath or respiratory aerosol of a subject. In an embodiment, the respiratory pathogen is a coronavirus. In an embodiment, the coronavirus is selected from the group consisting of SARS-COV virus, SARS-COV-2 virus, MERS-COV virus, OC43 virus, NL63 virus, 229E virus, and NKU1 virus. In a preferred embodiment, the respiratory pathogen is SARS-COV-2. In an embodiment, the cartridge, point-of-care or handheld breathalyzer system, and methods of the invention comprises or additionally comprises forward and reverse primers and exonuclease probe of Table 1A. Said primers and probe of Table 1A can be used to detect presence of SARS-COV-2 virus or viral genome in the sample.

The cartridge, point-of-care or handheld breathalyzer system, and methods of the invention can be used to not only detect intact respiratory pathogen, such as intact bacterium or viral particle, but may be used to detect genomic DNA originating or released from an inactive or fractured respiratory pathogen in breath or respiratory aerosol collected from a subject. The subject may be an animal. In an embodiment, the subject is a mammal. In a preferred embodiment, the subject is a human.

TABLE 1
(A) Listed are the forward primer, reverse primer and the exonuclease probe
for the RT-RPA-exonuclease probe reaction targeting the nucleocapsid region of the SARS-
CoV-2 genome. (B) Listed are the forward primer, reverse primer, DNA synthetic template
and molecular beacon probe that were used to test the RT-RPA-T7-Molecular beacon reaction.
The forward primer includes the T7 promoter site sequence at the 5′ end of the primer to enable
T7 RNA runoffs of the RPA amplicons, which facilitates binding of the molecular beacon
probe as there is limited competition for the targeted strand of RNA.
Table 1A
RT-RPA-Exonuclease Probe Reaction

| Description | Sequence |
|---|---|
| Forward | TCCCACCAACAGAGCCTAAAAAGGACAAAAAG |
| Reverse | TTAGGCCTGAGTTGAGTCAGCACTGCTCAT |
| Exonuclease Probe | CAGAAGAAACAGCAAACTGTGACTCTTC[T(FAM)]T[dSpacer]C\|T(BHQ-1)]GCTGCAGATTTGG[3-Sp3] |

Table 1B
T7-RT-RPA-Molecular Beacon Probe Reaction

| Description | Sequence |
|---|---|
| Forward | TAATACGACTCACTATAGGGAGAGTGTTCAAAGCAGGCGCA |
| Reverse | AGAAACCAACAAAATAGAACCATGCGTCCT |
| MB Probe | /S6-FAM/GCGCACTTCCATGCTAACAGATTCAAGGGTGCGC/38HQ.1/ |
| Template | AGTAAATGAGAGTGTTCAAAGCAGGCGCACGCTTGAATCTGTTAGCATGGAATAATAGAATAGGACGCATGGTTCTAT |
| | TTTGTTGGTTTCTAGGACCATC |

### REFERENCES

1. Pan M, Lednicky JA, Wu CY. Collection, particle sizing and detection of airborne viruses. J Appl Microbiol. 2019 December; 127 (6): 1596-1611. doi: 10.1111/jam.14278. Epub 2019 Jun. 26. PMID: 30974505; PMCID: PMC7167052.
2. Chao CYH, Wan MP, Morawska L, Johnson GR, Ristovski ZD, Hargreaves M, Mengersen K, Corbett S, Li Y, Xie X, Katoshevski D. Characterization of expiration air jets and droplet size distributions immediately at the mouth opening. J Aerosol Sci. 2009 February;40 (2): 122-133. doi: 10.1016/j.jaeros ci.2008.10.003. Epub 2008 Nov. 7. PMID: 32287373; PMCID: PMC7126899.
3. Ladhani Laila, Pardon Gaspard, Moons Pieter, Goossens Herman, van der Wijngaart Wouter. Electrostatic Sampling of Patient Breath for Pathogen Detection: A Pilot Study. Frontiers in Mechanical Engineering. 2020;vol 6; pg 40.

DOI==10.3389/fmech.2020.00040. ISSN=2297-3079
4. Li J, Macdonald J, von Stetten F. Review: a comprehensive summary of a decade development of the recombinase polymerase amplification. Analyst. 2018 Dec. 17;144 (1): 31-67. doi: 10.1039/c8an01621f. Erratum in: Analyst. 2020 Mar. 2;14S (5): 1950-1960. PMID: 30426974.
5. Zaneeta Dhesi, Virve I. Enne, Justin O'Grady, Vanya Gant, and David M. Livermore. Rapid and Point-of-Care Testing in Respiratory Tract Infections: An Antibiotic Guardian?. ACS Pharmacol. Transl. Sci. 2020, 3. 3, 401-417. Publication Date: May 12, 2020.
6. Leung NHL, Chu DKW, Shiu EYC, Chan KH, McDevitt JJ, Hau BJP, Yen HL. Li Y.

Ip DKM, Peiris JSM, Seto WH, Leung GM, Milton DK, Cowling B. J. Respiratory virus shedding in exhaled breath and efficacy of face masks. Nat Med. 2020 May: 26 (5): 676-680. doi: 10.1038/s41591-020-0843-2. Epub 2020 Apr. 3. Erratum in: Nat Med. 2020 May 27;: PMID: 32371934.
7. Jing Yan, Michael Grantham, Jovan Pantelic, P. Jacob Bueno de Mesquita, Barbara Albert. Fengjie Liu, Shery! Ehrman, Donald K. Milton, Aerosol shedding of infectious influenza virus. Proceedings of the National Academy of Sciences January 2018. 11S (5) 1081-1086: DOI: 10.1073/pnas.1716561115
8. Li, Hongying & Leong, Fong & Xu, George & Kang, Chang-Wei & Lim. Keng & Tan, Ban & Loo, Chian. (2021). Airborne dispersion of droplets during coughing; a physical model of viral transmission. Scientific Reports. 11. 10.1038/841598-021. 84245-2.
9. Daher RK, Stewart G, Boissinot M, Bergeron MG. Recombinase Polymerase Amplification for Diagnostic Applications. Clin Chem. 2016 July;62 (7): 947-58. doi: 10.1373/clinchem.2015.245829. Epub 2016 May 9. PMID: 27160000; PMCID: PMC7108464
10. Burcu Özay, Stephanie E McCalla. A review of reaction enhancement strategies for isothermal nucleic acid amplification reactions, Sensors and Actuators Reports.

### Volume 3, 2021, 100033, ISSN 2666-0539.

11. Amer HM. Abd B Wahed A, Sbalaby MA, Almajhdi FN, Hufert FT, Weidmann M. A new approach for diagnosis of bovine coronavirus using a reverse transcription recombinase polymerase amplification assay. J Virol Methods. 2013 November: 193 (2): 337. 40. doi: 10.1016/j.jviromet.2013,06.027. Epub 2013 Jun. 28. PMID: 23811231; PMCID: PMC7113639.
12. L. au YL, Ismail ib, Mustapa Nib, Lai MY, Tuan Soh TS, Haji Hassan A, et al. (2021) Development of a reverse transcription recombinase polymerase amplification assay for rapid and direct visual detection of Severe Acute Respiratory Syndrome Coronavirus 2 (SARS-COV-2). PLOS ONE 16 (J): e0245164.
13. Crannell ZA, Rohrman B, Richards-Kortum R. Development of a quantitative recombinase polymerase amplification assay with an internal positive control. J Vis Exp. 2015 Mar. 30; (97): 52620. doi: 10.3791/52620. PMID: 25867513; PMCID: PMC4401391.

1. A sample and assay breathalyzer cartridge for sample collection and performance of assay composed of multiple layers comprising:
a. a first layer comprising:
i. one or more blister pack(s) or flexible packages for holding liquids that with applied pressure rupture to release the liquid into the cartridge fluidics layer;
ii. an aperture for attachment of a tube or conduit to guide sample flow of entry, or a side wall for mating with a U-shaped clip comprising an aperture for attachment of a tube or conduit to guide sample flow or entry;
iii. one or more pressure or vacuum port(s) to mate with an actuator-detector-reader device, or pressure or vacuum ports as shown in FIGS. 2 and 3; and
iv. an exit vent for fluid flow;
b. a second or fluidic layer comprising:
i. one or more reservoir(s) to hold liquid from ruptured blister pack(s) from I. a.i and a lyophilized reagent(s);
ii. a network of fluidic channels, chambers and reservoirs to direct liquid flow within the network or a network of fluidic channels, chambers and reservoirs to direct fluid flow within the network as shown in FIG. 3 as items C through I and the associated interconnecting pathways and channels;
iii. moisture lock(s) to maintain all liquids and reagents within the cartridge and to vent air as illustrated in FIG. 2 and FIG. 3K:
iv. one or more optical window(s) to transmit light waves for fluorescent excitation and emission, or alternatively, one fluorescent excitation window and one emission window perpendicular to each other wherein the excitation window comprises a side wall of chamber I and emission window comprises ceiling of chamber I as shown in FIG. 3;
v. a stir bar(s) to ensure mixing of reagents, or a stir bar(s) to ensure mixing of reagents wherein chamber I and/or H in FIG. 3 comprises a stir bar;
vi. a filter membrane or matrix to capture breath respiratory aerosols, or a filter membrane or matrix to capture breath respiratory aerosols wherein the filter membrane is located in chamber C of FIG. 3; and
c. a third layer comprising:
i. one or more chambers with absorbent pad(s) or matrix, or one or more chambers with absorbent pad(s) or matrix as depicted in FIG. 2.

2. The cartridge of claim 1, wherein the sample is a respiratory aerosol, aerosol, liquid or fluid.

3. The cartridge of claim 1, wherein the assay comprises isothermal nucleic acid amplification and detection of a pathogen.

4. The cartridge of claim 1, wherein the isothermal nucleic acid amplification and detection of a pathogen comprise primers directed to bioinformatically significant region(s) of the pathogen's nucleic acid sequence for isothermal amplification and detection.

5. The cartridge of claim 4, wherein the primers directed to bioinformatically significant region(s) comprises nucleic acid sequence(s) specific for amplification of the pathogen's nucleic acid sequence.

6. The cartridge of claim 4, wherein the primers directed to bioinformatically significant region(s) comprises nucleic acid sequence(s) that selectively amplifies the pathogen's nucleic acid sequence over other nucleic acid sequences.

7. The cartridge of claim 4, where in the primers additionally comprises a promoter sequence for an RNA polymerase to bind. start transcription and generate single stranded nucleic acid strands for probe binding.

8. The cartridge of claim 7, wherein the promoter sequence is selected from the group consisting of T7 RNA polymerase promoter sequence, SP6 RNA polymerase promoter sequence, T3 RNA polymerase promoter sequence and an equivalent.

9. The cartridge of claim 1, wherein the first layer additionally comprises an opening for the emission window of the fluidics layer above a reaction chamber for measuring fluorescent intensities.

10. The cartridge of claim 1, additionally comprising one or more adhesive layers.

11. The cartridge of claim 10, wherein the adhesive layer adheres waste layer and fluidics layer.

12. The cartridge of claim 10, wherein the adhesive layer adheres fluidics layer and top layer.

13. The cartridge of claim 10, wherein the adhesive layer covering the emission window is optically compatible with fluorescence detection.

14. The cartridge of claim 1.b.ii, wherein the network of fluidic channels, chambers and reservoirs is a single network.

15. (canceled)

16. A sample and assay breathalyzer cartridge for detection of a respiratory pathogen(s) in the breath or respiratory aerosol of a subject comprising the sample and assay breathalyzer cartridge of claim 1 and forward and reverse primers that target a nucleic acid sequence unique to the pathogen or shared by a set of said pathogens

17. The cartridge of claim 16, wherein the respiratory pathogen(s) is a bacterium and/or a virus.

18. The cartridge of claim 17, wherein the bacterium and/or virus is viable, intact or infectious.

19. The cartridge of claim 17, wherein the bacterium and/or virus is not viable or not intact or exudes its genomic nucleic acid.

20. The cartridge of claim 19, wherein the genomic nucleic acid is RNA or DNA,

21. The cartridge of claim 17, wherein the bacterium is selected from the group of Streptococcus pneumoniae, Staphylococcus aureus, Methicillin-resistant Siphylococcus aureus (MRSA). Escherichia coli, Klebsiella pneumoniae, Pseudomonas aeruginosa, Acinetobacter baumannii, Stenotrophomonas maltophilia. Haemophilus influenzae, Legionella pneumophila. Mycoplasma Pneumonia, Chlamydia pneumonia, Mycobacterium tuberculosis and any bacterium that may be present in a breath or respiratory aerosol of a subject.

22-84. (canceled)
